(19) United States
(12) Patent Application Publication
(10) Pub. No.: US 2025/0308700 A1
(43) Pub. Date: Oct. 2, 2025

(54) RISK PREDICTION METHOD AND DEVICE OF PREGNANT WOMEN SUFFERING FROM GESTATIONAL DIABETES MELLITUS BASED ON MACHINE LEARNING

(71) Applicant: THE FIRST AFFILIATED HOSPITAL OF XIAMEN UNIVERSITY, XIAMEN (CN)

(72) Inventors: MIN ZHAO, XIAMEN (CN); LIHONG HUANG, XIAMEN (CN); JINGKAI ZENG, XIAMEN (CN)

(21) Appl. No.: 18/618,219

(22) Filed: Mar. 27, 2024

(51) Int. Cl. G16H 50/30; G06V 10/82; G06V 20/68; G08B 21/18; G16H 20/60; G16H 50/20

(52) U.S. Cl. CPC G16H 50/30; G06V 10/82; G06V 20/68; G08B 21/182; G16H 20/60; G16H 50/20

(57) ABSTRACT

The present disclosure provides a risk prediction method and a risk prediction device of the pregnant women suffering from the gestational diabetes mellitus based on machine learning. The sugar intake is obtained by processing the food images of the pregnant women before eating through the convolutional neural network model. Then the average daily sugar intake is obtained based on the sugar intake. Finally, based on the average daily sugar intake and the physiological indicators of the pregnant women, the risk degree of the pregnant women suffering from the gestational diabetes mellitus is obtained by processing though the deep neural network model, thereby accurately predicting the risk degree of the pregnant women suffering from the gestational diabetes mellitus. Therefore, the medical nutrition management can be carried out in time for the pregnant women before they suffer from the gestational diabetes mellitus, thereby reducing the occurrence of the gestational diabetes mellitus.

### TECHNICAL FIELD

The present disclosure relates to the technical field of machine learning, and in particular to a risk prediction method and a risk prediction device of the pregnant women suffering from the gestational diabetes mellitus based on machine learning.

### BACKGROUND

Gestational diabetes mellitus is the disease with the highest incidence rate during pregnancy. Gestational diabetes mellitus is a group of metabolic diseases characterized by hyperglycemia in the second trimester of pregnancy (24 to 28 weeks), which not only leads to fetal macrosomia, neonatal hypoglycemia, dystocia and postpartum hemorrhage, but also increases the possibility of cardiovascular diseases and metabolic disorders in the future for both mothers and children. However, at present, there is still a lack of screening methods and diagnostic criteria for gestational diabetes mellitus in early pregnancy, which is easy to miss diagnosis and delay treatment. At present, at home and abroad, most of the oral glucose tolerance test (OGTT) from 24 weeks to 28 weeks is used to diagnose gestational diabetes mellitus, and at this time, the adverse effects of hyperglycemia on both mothers and children have been produced. Therefore, early screening and early intervention of the pregnant women suffering from the gestational diabetes mellitus is the key link to prevent the occurrence of gestational diabetes mellitus, reduce physical injury and improve pregnancy outcomes.

Therefore, how to accurately predict the risk of gestational diabetes mellitus in the early stage of pregnancy is an urgent problem to be solved.

### SUMMARY OF THE DISCLOSURE

The present disclosure mainly solves the technical problem that how to accurately predict the risk of the pregnant women suffering from the gestational diabetes mellitus in the early stage of pregnancy.

In a first aspect, one embodiment of the present disclosure provides a risk prediction method of the pregnant women suffering from the gestational diabetes mellitus based on machine learning, and the method includes: S1, obtaining food images of pregnant women before eating; S2, processing the food images of the pregnant women before eating based on a convolutional neural network model, to obtain sugar intake, wherein an input of the convolutional neural network model includes the food images of the pregnant women before eating, and an output of the convolutional neural network model is the sugar intake; S3, obtaining total sugar intake over a period of time based on a plurality of amounts of the sugar intake over the period of time; S4, determining average daily sugar intake based on the total sugar intake over the period of time; and S5, determining a risk degree of the pregnant women suffering from the gestational diabetes mellitus by using a deep neural network model based on the average daily sugar intake and physiological indicators of the pregnant women, wherein an input of the deep neural network model includes the average daily sugar intake and the physiological indicators of the pregnant women, and an output of the deep neural network model is the risk degree of the pregnant women suffering from the gestational diabetes mellitus.

In some embodiments, the obtaining food images of pregnant women before eating, includes: photographing food of pregnant women before eating based on a mobile phone to obtain the food images.

In some embodiments, the convolutional neural network model is obtained by training using a gradient descent method.

In some embodiments, a warning prompt is issued when the average daily sugar intake is greater than a first threshold.

In some embodiments, a warning prompt is issued when the average daily sugar intake is less than a second threshold.

In some embodiments, the physiological indicators of the pregnant women include a body mass index, whether to take folic acid, a menarche age, a hemoglobin value, a leukocyte value, a platelet value, a serum creatinine value, a hepatitis B virus value, a hepatitis B virus surface antigen value, a serum alanine aminotransferase value, an albumin value, and a total bilirubin value.

In some embodiments, the training process includes: obtaining a plurality of training samples, wherein the training samples includes sample input data and labels corresponding to the sample input data, the sample input data is a sample food image, and the label is the sugar intake corresponding to the sample food image; and training an initial convolutional neural network model based on the plurality of training samples, to obtain the convolutional neural network model.

In a second aspect, one embodiment of the present disclosure provides a computer program product including a computer program. When the computer program is executed by a processor, operations of the risk prediction method of pregnant women suffering from gestational diabetes mellitus based on machine learning are implemented.

In a third aspect, one embodiment of the present disclosure provides an electronic device. The electronic device includes a memory, a processor, and a computer program. The computer program is stored in the memory and configured to be executed by the processor to implement the above methods.

In a fourth aspect, one embodiment of the present disclosure provides a computer-readable storage medium. The program is stored in the computer-readable storage medium, and can be executed by the processor to implement a method according to any one of the above aspects.

According to the risk prediction method and the risk prediction device of the pregnant women suffering from the gestational diabetes mellitus based on machine learning provided by the above embodiments, the sugar intake is obtained by processing the food images of the pregnant women before eating through the convolutional neural network model. And then the average daily sugar intake is obtained based on the sugar intake. Finally, based on the average daily sugar intake and the physiological indicators of the pregnant women, the risk degree of the pregnant women suffering from the gestational diabetes mellitus is obtained by processing though the deep neural network model, so as to accurately predict the risk degree of the pregnant women suffering from the gestational diabetes mellitus. Therefore, the medical nutrition management can be carried out in time for the pregnant women before they suffer from the gestational diabetes mellitus, so as to reduce the occurrence of the gestational diabetes mellitus.

### BRIEF DESCRIPTION OF THE DRAWINGS

FIG. 1 is a flow view of a risk prediction method of the pregnant women suffering from the gestational diabetes mellitus based on machine learning in some embodiments of the present disclosure.

FIG. 2 is a structural schematic view of a risk prediction device of the pregnant women suffering from the gestational diabetes mellitus based on machine learning in some embodiments of the present disclosure.

FIG. 3 is a schematic view of an electronic device in some embodiments of the present disclosure.

### DETAILED DESCRIPTION

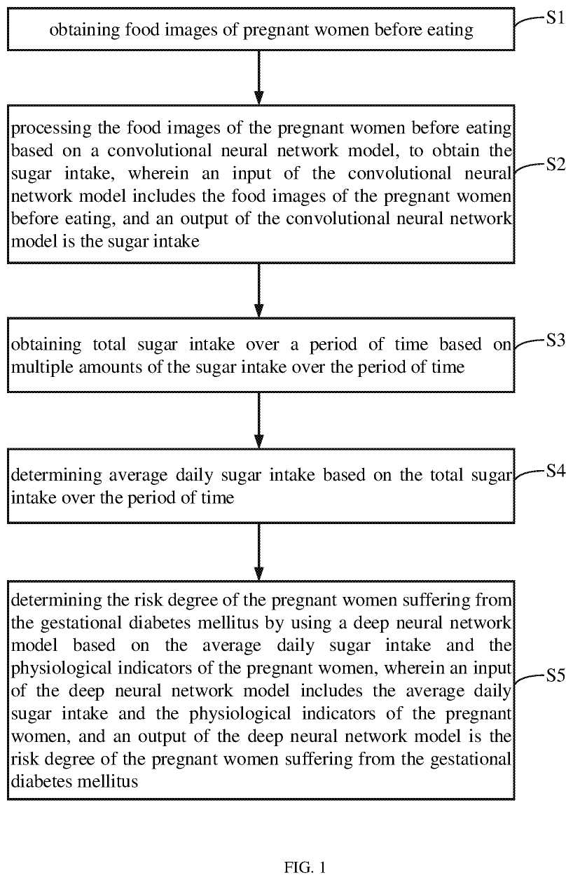

In the embodiments of the present disclosure, as illustrated in FIG. 1, a risk prediction method of the pregnant women suffering from the gestational diabetes mellitus based on machine learning is provided, and the method includes the following operations S1 to S5.

At the operation S1, the method may include obtaining food images of pregnant women before eating.

Before the pregnant women eat each time, the food that the pregnant women eat can be photographed by using the mobile phone, to obtain the food images. The food images can be video data or images. The video data refers to dynamic images recorded in the form of electrical signals, which is composed of multiple time-continuous static images. Each static image is one frame of the video data. In some embodiments, the video data at one time point may include multiple static images.

In some embodiments, a format of the video data may include but is not limited to, one or more combinations of a High Density Digital Video Disc (DVD), a Flash Video (FLV), a Motion Picture Experts Group (MPEG), an Audio Video Interleaved (AVI), a Video Home System (VHS), and a Real Media file format (RM), etc.

The food images contain the shape, size, and type of food. The food images can be used for image recognition to obtain the sugar content of food. The cause of the gestational diabetes mellitus is closely related to the sugar intake of the pregnant women, the higher the sugar intake in early pregnancy, the higher the probability of the gestational diabetes mellitus. Because it is difficult to control the sugar intake of the pregnant women, the food images of the pregnant women before eating are photographed before every time the pregnant women eat, and the sugar intake of each time is obtained by image recognition of the food images, so that the risk of the gestational diabetes mellitus can be predicted based on the sugar intake.

Gestational diabetes mellitus (GDM) is diabetes that occurs during pregnancy. The gestational diabetes mellitus brings a greater physiological burden to the pregnant women. Long-term hyperglycemia is likely to lead to ketoacidosis in the pregnant women, excessive amniotic fluid, premature rupture of membranes and premature delivery, and also lead to abnormal embryonic development and even death. The incidence of abortion is up to 15%-30%.

At the operation S2, the method may include processing the food images of the pregnant women before eating based on a convolutional neural network model, to obtain the sugar intake. An input of the convolutional neural network model includes the food images of the pregnant women before eating, and an output of the convolutional neural network model is the sugar intake.

The sugar intake represents the amount of sugar in the food at each meal. The sugars in the sugar content include sucrose, fructose, glucose, maltose, lactose, glycogen, etc. Excessive sugar intake is likely to lead to the occurrence of the gestational diabetes mellitus. For example, the sugar intake can be 1 gram, 2 grams, 3 grams, 5 grams, 10 grams, etc.

The convolutional neural network model includes a convolutional neural network. The convolutional neural network (CNN) can be multi-layer neural networks (e.g., including at least two layers). The at least two layers may include at least one of a convolutional layer (CONV), a rectified linear unit (ReLU) layer, a pooling layer (POOL), or a fully connected layer (FC). The at least two layers of the convolutional neural network (CNN) can correspond to neurons arranged in three dimensions: width, height, and depth. In some embodiments, the convolutional neural network (CNN) may have an architecture of [input layer-convolutional layer-rectified linear unit layer-pooling layer-fully connected layer]. The convolutional layer can calculate the output of neurons connected to a local region in the input, and calculate the dot product between the weight of each neuron and its small region connected in the input volume.

The convolutional neural network model can process and recognize the sugar intake based on the food images of the pregnant women before eating. The input of the convolutional neural network model includes the food images of the pregnant women before eating, and the output of the convolutional neural network model is the sugar intake.

The convolutional neural network model can be obtained through training multiple training samples. The training sample includes sample input data and labels corresponding to the sample input data. The sample input data in the training sample includes sample food images. The output label in the training sample is the sugar intake corresponding to the sample food images. Multiple groups of training samples can be manually annotated by the staff. For example, the staff can manually annotate the sample food images, annotate the sugar intake corresponding to the sample food images, and use the annotated sugar intake as the output label of the training samples for training. In some embodiments, the initial convolutional neural network model can be trained by using a gradient descent method to obtain the trained convolutional neural network model. Specifically, based on the training samples, the loss function of the convolutional neural network model is constructed; and the parameters of the convolutional neural network model are adjusted through the loss function of the convolutional neural network model, until the loss function value converges or is less than the preset threshold, and the training is completed. The loss function can include but is not limited to logarithmic (log) loss function, squared loss function, exponential loss function, Hinge loss function, and absolute value loss function, etc.

At the operation S3, the method may include obtaining total sugar intake over a period of time based on multiple amounts of the sugar intake over the period of time.

After the convolutional neural network model identifies the sugar intake, multiple amounts of the sugar intake over the period of time can be added together to obtain the total sugar intake over the period of time. The period of time can be 1 day, 2 days, 7 days, or one month, etc.

At the operation S4, the method may include determining average daily sugar intake based on the total sugar intake over the period of time.

For example, the average daily sugar intake can be obtained by dividing the total sugar intake over the period of time by the total number of days over that period. For example, when the total sugar intake is 100 grams and the total number of days in a period is 2 days, the total sugar intake that is 100 grams is divided by the total number of days in a period that is 2 days, and the average daily sugar intake is 50 grams.

In some embodiments, when the average daily sugar intake is greater than a first threshold, a warning prompt is issued. In some embodiments, the first threshold may be 50 grams.

In some embodiments, too low sugar intake can lead to poor physical strength, hypoglycemia, and endocrine disorders. Therefore, when the average daily sugar intake is less than a second threshold, a warning prompt is issued. In some embodiments, the second threshold may be 5 grams.

At the operation S5, the method may include determining the risk degree of the pregnant women suffering from the gestational diabetes mellitus by using a deep neural network model based on the average daily sugar intake and the physiological indicators of the pregnant women. An input of the deep neural network model includes the average daily sugar intake and the physiological indicators of the pregnant women, and an output of the deep neural network model is the risk degree of the pregnant women suffering from the gestational diabetes mellitus.

Since the determination of the risk degree of the pregnant women suffering from the gestational diabetes mellitus is related to the influence of various factors, the average daily sugar intake and the physiological indicators of the pregnant women are taken as the inputs of the deep neural network model, so that the output risk degree of the pregnant women suffering from the gestational diabetes mellitus is more accurate.

The physiological indicators of the pregnant women include a body mass index, whether to take folic acid, a menarche age, a hemoglobin value, a leukocyte value, a platelet value, a serum creatinine value, a hepatitis B virus value, a hepatitis B virus surface antigen value, a serum alanine aminotransferase value, an albumin value, and a total bilirubin value.

Body Mass Index (BMI) is a commonly used international standard to measure how fat and thin is person is and whether they are healthy. Whether to take folic acid includes two situations: taking folic acid and not taking folic acid. Folic acid is a water-soluble vitamin with the molecular formula C19H19N7O6. The menarche age refers to the age at which a woman has her first menstrual vaginal bleeding. The hemoglobin value can be 110-150 g/L, 120-160 g/L, or 170-200 g/L.

The risk degree of the pregnant women suffering from the gestational diabetes mellitus indicates the risk of the pregnant women suffering from the gestational diabetes mellitus. For example, the risk degree of the pregnant women suffering from the gestational diabetes mellitus can be a number between 0 and 1. For example, the risk degree of the pregnant women suffering from the gestational diabetes mellitus is 0.4, which means that the pregnant women have a 40% chance of suffering from the gestational diabetes mellitus. For another example, the risk degree of the pregnant women suffering from the gestational diabetes mellitus is 0.1, which means that the pregnant women have a 10% chance of suffering from the gestational diabetes mellitus. For another example, the risk degree of the pregnant women suffering from the gestational diabetes mellitus can be high risk, medium risk, and low risk. In some embodiments, when the deep neural network model determines that the risk degree of the pregnant women is high, for example, the risk level is greater than 0.3 or the risk degree is high risk, doctors can carry out corresponding interventions based on the risk level to avoid the occurrence of the gestational diabetes mellitus, such as reminding the pregnant women to pay attention to exercise, eat light, strictly control sugar intake, etc. In some embodiments, when the deep neural network model determines that the risk level of the pregnant women is moderate, for example, when the risk degree is greater than 0.1 and less than 0.3, or when the risk degree is moderate, doctors can carry out corresponding interventions based on the risk level to avoid the occurrence of the gestational diabetes mellitus, such as reminding pregnant women to eat less and eat more meals, slightly control sugar intake, etc. In some embodiments, when the deep neural network model determines that the risk level of the pregnant women is low, for example, when the risk degree is less than 0.1 or the risk degree is low, no intervention is temporarily taken.

In some embodiments, the risk degree of the pregnant women suffering from the gestational diabetes mellitus can be determined through the trained deep neural network model. The deep neural network model includes a deep neural network. The deep neural network can include multiple processing layers, each processing layer is composed of multiple neurons, and each neuron performs a matrix transformation on the data. The parameters used in the matrix can be obtained through training. The deep neural network model can also be any existing neural network model that is capable of processing multiple features, such as RNN, CNN, DNN, etc. The deep neural network model can also be a customized model according to requirements. The input of the deep neural network model is the average daily sugar intake and the physiological indicators of the pregnant women. The output of the deep neural network model is the risk degree of the pregnant women suffering from the gestational diabetes mellitus.

The deep neural network model can be obtained through training samples. The training samples include sample input data and the output labels corresponding to the sample input data. The sample input data of the training sample are the daily sugar intake of the sample and the physiological indicators of the sample pregnant women. The output label of the training sample is the risk degree of the pregnant women suffering from the gestational diabetes mellitus. Multiple groups of training samples can be obtained by labeling historical data, for example, the daily sugar intake of the pregnant women, the physiological indicators of the pregnant women, and the data whether the pregnant women eventually suffer from the gestational diabetes mellitus in the historical data are extracted. The output label is obtained by manually labeling the daily sugar intake of the pregnant women and the risk degree corresponding to the physiological indicators of the pregnant women by doctors. In some embodiments, the deep neural network model can be trained by using the gradient descent method to obtain the trained deep neural network model. Specifically, based on the training samples, the loss function of the deep neural network model is constructed; and the parameters of the deep neural network model are adjusted through the loss function of the deep neural network model, until the loss function value converges or is less than the preset threshold, and the training is completed. The loss function can include but is not limited to logarithmic (log) loss function, squared loss function, exponential loss function, Hinge loss function, and absolute value loss function, etc.

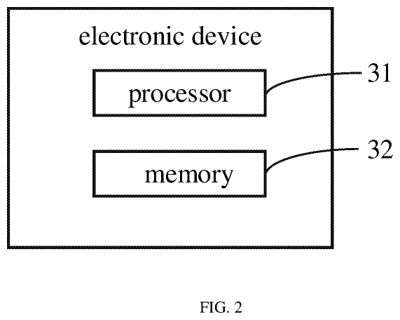

Based on the same invention concept, the embodiments of the present disclosure provide an electronic device as shown in FIG. 2. The electronic device includes a processor 31 and a memory 32.

The memory 32 is configured for storing program instructions executed by the processor 31. The processor 31 is configured to implement the risk prediction method of the pregnant women suffering from the gestational diabetes mellitus based on the machine learning as provided above.

Based on the same inventive concept, the embodiments of the present disclosure provide a non-temporary computer-readable storage medium. When the instructions in the storage medium are executed by the processor 31 of the electronic device, the electronic device can execute the risk prediction method of the pregnant women suffering from the gestational diabetes mellitus based on the machine learning as provided above provided above.

Based on the same inventive concept, the embodiments of the present disclosure also provide a computer program product. When a computer program is executed by the processor, the risk prediction method of the pregnant women suffering from the gestational diabetes mellitus based on the machine learning as provided above can be implemented.

The above description is only preferred embodiments of the present disclosure and is not intended to limit the present disclosure. For those skilled in the art, the present disclosure may have various modifications and variations. Any modification, equivalent replacement, improvement, etc. made in the spirit and principle of the present disclosure should be included in the scope of protection of the present disclosure.

What is claimed is:

1. A risk prediction method of pregnant women suffering from gestational diabetes mellitus based on machine learning, comprising:
S1, obtaining food images of pregnant women before eating;
S2, processing the food images of the pregnant women before eating based on a convolutional neural network model, to obtain sugar intake, wherein an input of the convolutional neural network model comprises the food images of the pregnant women before eating, and an output of the convolutional neural network model is the sugar intake;
S3, obtaining total sugar intake over a period of time based on a plurality of amounts of the sugar intake over the period of time;
S4, determining average daily sugar intake based on the total sugar intake over the period of time; and
S5, determining a risk degree of the pregnant women suffering from the gestational diabetes mellitus by using a deep neural network model based on the average daily sugar intake and physiological indicators of the pregnant women, wherein an input of the deep neural network model comprises the average daily sugar intake and the physiological indicators of the pregnant women, and an output of the deep neural network model is the risk degree of the pregnant women suffering from the gestational diabetes mellitus.

2. The risk prediction method of pregnant women suffering from gestational diabetes mellitus based on machine learning according to claim 1, wherein the obtaining food images of pregnant women before eating, comprises: photographing food of pregnant women before eating based on a mobile phone to obtain the food images.

3. The risk prediction method of pregnant women suffering from gestational diabetes mellitus based on machine learning according to claim 1, further comprising: obtaining the convolutional neural network model by training using a gradient descent method.

4. The risk prediction method of pregnant women suffering from gestational diabetes mellitus based on machine learning according to claim 1, further comprising: issuing a warning prompt when the average daily sugar intake is greater than a first threshold.

5. The risk prediction method of pregnant women suffering from gestational diabetes mellitus based on machine learning according to claim 1, further comprising: issuing a warning prompt when the average daily sugar intake is less than a second threshold.

6. The risk prediction method of pregnant women suffering from gestational diabetes mellitus based on machine learning according to claim 1, wherein the physiological indicators of the pregnant women comprise a body mass index, whether to take folic acid, a menarche age, a hemoglobin value, a leukocyte value, a platelet value, a serum creatinine value, a hepatitis B virus value, a hepatitis B virus surface antigen value, a serum alanine aminotransferase value, an albumin value, and a total bilirubin value.

7. The risk prediction method of pregnant women suffering from gestational diabetes mellitus based on machine learning according to claim 1, wherein the convolutional neural network model is obtained through a training process, and the training process comprises:
obtaining a plurality of training samples, wherein the training samples comprises sample input data and labels corresponding to the sample input data, the sample input data is a sample food image, and the label is the sugar intake corresponding to the sample food image; and
training an initial convolutional neural network model based on the plurality of training samples, to obtain the convolutional neural network model.

8. A computer program product, comprising:
a computer program, wherein when the computer program is executed by a processor, operations of the risk prediction method of pregnant women suffering from gestational diabetes mellitus based on machine learning according to any one of claims 1 to 7 are implemented.

9. An electronic device, comprising:
a memory;
a processor; and
a computer program, stored in the memory and configured to be executed by the processor to implement operations in the risk prediction method of pregnant women suffering from gestational diabetes mellitus based on machine learning according to any one of claims 1 to 7.
